(19) United States
(12) Patent Application Publication
(10) Pub. No.: US 2002/0019705 A1
(43) Pub. Date: Feb. 14, 2002

(54) System and method for analysis of genetic networks

(76) Inventors: Stuart A. Kauffman, Santa Fe, NM (US); Bruce K. Sawhill, Santa Fe, NM (US); Roumen Borissov, Somerville, MA (US)

Correspondence Address: PENNIE & EDMONDS LLP, 1667 K STREET NW, SUITE 1000, WASHINGTON, DC, 20006

(21) Appl. No.: 09/838,156

(22) Filed: Apr. 20, 2001

Related U.S. Application Data
(63) Continuation of application No. PCT/US99/24658, filed on Oct. 21, 1999.
(60) Provisional application No. 60/105,075, filed on Oct. 21, 1998.

(51) Int. Cl. G01N 33/48; G01N 33/50

(52) U.S. Cl. 702/19; 702/20

(57) ABSTRACT

The present invention relates to experimental and algorithmic methods for analysis of genetic regulatory networks. More particularly, the present invention provides methods of partitioning genes withing an organism into a plurality of groups and identification and characterization of correlations between genes and groups of genes in the genomic networks of real viruses, cells, and tissues.

This application claims the benefit under 35 U.S.C. §119(e) of provisional application number 60/105,075, filed on Oct. 21, 1998, which is hereby incorporated by reference in its entirety.

### 1. INTRODUCTION

The present invention relates to experimental and algorithmic methods for analysis of genetic regulatory networks. More particularly, the present invention provides methods of partitioning genes withing an organism into a plurality of groups and identification and characterization of correlations between genes and groups of genes in the genomic networks of real viruses, cells, and tissues.

### 2. BACKGROUND OF THE INVENTION

Living systems, including viruses, prokaryotes, and eukaryotes, possess genetic systems composed of structural genes and cis acting genes that serve to regulate the genetic activities of nearby structural genes, trans acting genes and trans acting factors, namely genes whose RNA or protein or other product, or other factors, bind singly or in complexes to cis acting sites to modulate the genetic activity of nearby structural genes. In addition, eukaryotic genomes contain exons and introns. Gene regulation involves transcription into RNA, in eukaryotes called heterogeneous nuclear RNA or hnRNA. In eukaroytes, the hnRNA is processed in a variety of ways to create mature messenger RNA, or mRNA, that is transported to the cytoplasm. In both prokaryotes and eukaryotes, mRNA in the cytoplasm is translated into proteins. Those proteins may be subjected to post translational modifications including cleavage, phosphorylation, and so forth, that modulate their biological activities.

The number of structural genes ranges from a few dozen in viruses to a few hundred to a few thousand in bacteria, to perhaps 15,000 in Drosophila, to 20,000 in some plants, to an estimated 80,000 to 100,000 in human cells.

Since the work of F. Jacob and I. Monod in 1961 and 1963, it has been clear that genes, via their products can increase or decrease the activity of other genes or turn other genes “on” or “off.” A gene is said to have been turned on if the activity of the gene increases from a lower level to a higher level. A gene is said to have been turned off if the activity of that gene decreases from a higher level to a lower or minimal level. Cells, in short, have a vast, parallel processing molecular genetic regulatory network of the kinds of genes and their products noted above, whose joint dynamical activity within and between cells underlies both normal ontogeny and much of pathogenesis, ranging from viral infections to cancer to metaplasias, to tissue degeneration and regeneration.

Understanding the coordinated behavior of the genetic regulatory network in cells has emerged as among the most important problems in molecular, cell, and developmental biology as well as in biomedicine. It is almost certainly true that a “post-genomic” medicine will be one that learns to manipulate patterns of gene network activities within and between cells to treat or prevent disease.

Genetic regulatory networks (GRN) model systems of interdependent variables which change over time. A GRN comprises a plurality of variables, a system state defined as the value of the plurality of variables, and a plurality of regulatory rules corresponding to the plurality of variables which determine the next system state from previous system states. GRNs can model a wide variety of real world systems such as the interacting components of a company, the conflicts between different members of an economy and the interaction and expression of genes in cells and organisms.

Genes contain the information for constructing and maintaining the molecular components of a living organism. Genes directly encode the proteins which make up cells and synthesize all other building blocks and signaling molecules necessary for life. During development, the unfolding of a genetic program controls the proliferation and differentiation of cells into tissues. Since the function of a protein depends on its structure, and hence on its amino acid sequence and the corresponding gene sequence, the pattern of gene expression determines cell function and hence the cell's system state and the rules by which the state is changed.

In a GRN representing the interaction and expression of genes in cells and single-cell organisms, the variables represent the activation states of the genes. For example, the level of activity of a gene can be measured by the number of messenger RNA (mRNA) transcripts of the gene made per unit time or the number of proteins translated from the mRNAs per unit time. The regulatory rules are determined by the transcription regulatory sites next to each gene and the interactions between the gene products and these sites. Binding of molecules to these sites in various combinations and concentrations determines the degree of expression of the corresponding gene. Since these molecules are proteins or RNA's made by other genes, the network rules are functions of the activation states of the genes which they control. Genes are constantly exposed to varying concentrations of these controlling substances, so such a system can be considered as a GRN with an asynchronous, continuous time update rule.

As is well-known for all types of dynamical systems, these networks demonstrate attractors and basins of attraction. See Stephen E. Harris, Bruce K. Sawhill, Andrew Wuensche, and Stuart A. Kauffman, Biased eukaryotic gene regulation rules suggest genome behavior is near edge of chaos, Technical Report 97-05-039, Santa Fe Institute, 1997 (Harris et al.); Roland Somogyi and Caxol Ann Sniegoski, Modeling the complexity of genetic networks: Understanding multigenic and pleiotropic regulation, Complexity, 1(6):45-63, 1996; Staurt Kauffman, The Origins of Order, Oxford University Press, New York, 1993 (Origins of Order). An attractor is a state or set of states to which the system moves and then remains within for all future generations. Thus, an attractor is a recurrent pattern of states of system variables that typically occupies a sub-volume of the space containing all possible states of system variables. A basin of attraction is the set of states that eventually lead to a given attractor. In general, a system of N variables can have between 1 and 2^{N} attractors with basins ranging in size from the entire space of possible states of system variables to individual states.

In a non-limiting interpretation that guides some of the procedures of the present invention, different cell types of an organism are interpreted to correspond to different attractors in dynamic genetic network of the genes, cells, and cell types cells of that organism.

Identifying the GRN representing the interaction and expression of genes in a class of cells is of fundamental importance for medical diagnostic and therapeutic purposes. For example, normal and cancerous cells may have identical surface markers and surface receptors and can be difficult to distinguish with chemotherapeutic agents. A GRN model of the interaction and expression of genes in the cells can indicate functional differences between normal and cancerous cells that provide a basis for differentiation not dependent on cell surface markers. The GRN also provides a means to identify the receptors or genetic targets to which molecule design techniques such as combinatorial chemistry and high throughput screening should be directed to achieve given functional effects. Such techniques are frequently used now, and pharmaceutical and biotechnology companies suffer from uncertainty as to which targets and receptors are worthy of study. The approach described below can greatly assist in this process. See Gene Regulation and the Origin of Cancer: A New Method, A. Shah, Medical Hypothesis (1995) 45,398-402 and Cancer progression: The Ultimate Challenge, Renato Dubbecco, Int. J. Cancer: Supplement 4, 6-9 (1989).

### 3. SUMMARY OF THE INVENTION

The current invention lays out a set of comprehensive procedures, experimental and algorithmic, for the analysis of real genetic regulatory networks of biological systems. These novel procedures are based, in part, on published studies of model genetic networks as described above and reviewed in Origins of Order, by Kauffman and the Kauffman Ballivet U.S. patent application No. 09/165,794 filed Oct. 2 1998, by inventors Kauffman and Ballivet, each of which is incorporated herein by reference in its entirety.

A further aim of the present invention is to provide experimental and algorithmic means to identify isolated green islands in the genomic networks of real viruses, cells, and tissues.

The present invention provides a method for partitioning a plurality of genes into one or more groups comprising the steps of: selecting a first one of said genes and a second one of said genes; measuring a degree of correlation between said first gene and said second gene; and assigning said first gene and said second gene into a same one of said groups if said degree of correlation exceeds a predetermined threshold.

It is an aspect of the invention to provide a system for partitioning a plurality of genes into one or more groups comprising:

a programmed computer comprising a memory having at least one region storing computer executable program code and a processor for executing the program code stored in said memory, wherein the program code includes:

code to select a first one of said genes and a second one of said genes;

code to measure a degree of correlation between said first gene and said second gene; and

code to assign said first gene and said second gene into a same one of said groups if said degree of correlation exceeds a predetermined threshold.

It is an aspect of the invention to provide a method for partitioning a plurality of genes into one or more groups comprising the steps of:

defining a state for each of said genes;

selecting at least one of said genes;

initiating a perturbation on said selected gene to change said state of said selected gene;

identifying zero or more of said genes that experience a change in said state in response to said perturbation.

Methods for perturbing the physiological state of biological samples to effect different gene activation states are described in detail, below. Furthermore, methods for detecting gene expression of a plurality of genes in the biological samples, including expression levels resulting from such perturbations, are described in detail below. The information obtained from measuring, quantitatively or qualitatively, the level of expression of a plurality of genes in a biological sample can be used in accordance with the methods disclosed herein to identify and characterize genetic regulatory networks.

The identification and characterization of such genetic regulatory networks provides a characteristic “snapshot” of the physiological state of the biological sample. The information that constitutes these snapshots include characterization of the expression level of a plurality of genes that constitute a genetic regulatory network, or a sub-network within a given genetic regulatory network. These snapshots, therefore, are useful in designing approaches for identifying disease states and designing approaches for disease intervention. Therefore, the methods described herein are useful in disease diagnosis, identifying targets for therapeutic intervention, and monitoring the progress of therapeutic treatments. More particularly, the characteristic gene activation state of a diseased biological sample can be compared to that of a normal sample to provide a ready indicator of disease. Moreover, individual genes that are expressed at a different rate in a disease state as compared with a normal state are candidates for therapeutic modalities that alter their expression to approximate the expression level of the normal state. In addition, the progress of treatment regimens can be monitored by examining the gene activation state of biological sample of a subject at different stages of treatment. The effectiveness of the treatment is indicated by a progression of the gene expression pattern from the disease state to the normal state. Thus, treatment regimens can be optimized by correlating the treatment with a change in expression pattern that approaches that of the normal state.

### 4. BRIEF DESCRIPTION OF THE FIGURES

FIG. 1 is a plot of the expected distance between two states at time T+1 as a function of the normalized distance between the two states a moment earlier.

FIG. 2 is a plot of the log of the number of avalanches versus the log of the size of the avalanche produced by reversing the activity of a single randomly chosen gene within a model genetic network.

FIG. 3 is a histogram of the number of times a given mutual information was observed for pairs of genes within the same green islands of a model genetic network.

FIG. 4 is a histogram of the number of times a given mutual information was observed for pairs of genes within different green islands of a model genetic network.

FIG. 5 discloses a representative computer system in conjunction with which the embodiments of the present invention may be implemented.

### 5. DETAILED DESCRIPTION OF THE INVENTION

The present invention presents, without limitation, a number of experimental and algorithmic methods to establish whether eukaryotic cells are in the ordered regime, whether isolated green islands exist, to determine which genes are members of which isolated green islands, which genes are members of the red frozen structure, the regulatory connections and rules among the genes within each isolated green island, and the network more generally.

5.1 Numerical Models of Genetic Networks

A broad area of mathematical and algorithmic work has been carried out in which genes are modeled either as binary variables, e.g., “on-off” devices; as “piecewise linear” devices, or as “continuous sigmoidal” devices. See, Origins of Order, incorporated herein by reference. Broadly, the same results are obtained in all cases. Some of the results of these simulations are listed below and constitute some of the conceptual background for the present invention.

1) Parallel processing networks of thousands of genes and their products behave in two broad regimes: one, an ordered Regime or, two, a Chaotic regime.

2) A rough phase transition, often defined as the “edge of chaos”, separates these two regimes in the appropriate network parameter spaces.

3) Whether ordered or chaotic, all these model genetic networks are parallel processing non-linear dynamical systems. For deterministic models in all these classes, the generic dynamical behavior of a typical network breaks up the state space of possible combination of gene, RNA, and protein activities into one or more “basins of attraction”. Within each basin of attraction, the vector field, or transistions between discrete states, representing the dynamics of the system, yields trajectories that flow, rather like creeks flowing to a mountain lake, into a recurrent subset of the state space called an “attractor”. In continuous systems, the attractor might be a steady state, a limit cycle, a cjuasiperiodic orbit, or a chaotic “strange attractor”. In discrete deterministic synchronous state spaces the attractor is typically a “state cycle” around which the system orbits. The length, or number of states, on the state cycle can range from 1 to all the possible states.

If the network has more than one basin of attraction, then the system will flow to the attractor that “drains” the basin of attraction in which the network was initiated. The set of alternative attractors represent the alternative asymptotic behaviors of the network.

A variety of characteristics distinguish the ordered from the chaotic regime. See, Origins of Order, incorporated herein by reference. Briefly, in the ordered regime, the network generically flows from any initial state to an attractor. Initially, genes may turn on and off, e.g. increase or decrease in activity, e.g. expression levels, as may levels of their RNA and protein products, in complex temporal patterns. But, for binary synchronous networks, as the attractor is approached, the activities of more and more genes and their products become fixed in on or fixed in off values. For the piecewise linear or continuous sigmoidal networks, more and more genes and their products become essentially “fixed” at near minimal or near maximal activities. It is convenient to designate these fixed genes, variables, or products as “red”. Then in the ordered regime, typically, a red connected cluster of genes and products percolates or extends across the network. The technical definitions of percolates include the concepts that the size of the red “frozen” sea of fixed genes scales up in size with the size of the genetic network, and that in any such network there are connected regulatory pathways among the fixed genes along which all the genes on the pathway are fixed on or off. In the ordered regime, this “core” of the red frozen structure is the same for all the different attractors of the entire network. Thus, for example, if the genetic network has 200 different attractors, e.g. cell types, the red frozen core would be in substantially the same fixed state of activity, with perhaps small modulations, in all 200 attractors.

In the ordered regime, once the red frozen structure forms, isolated islands or groups of variables or genes and their products remain that may either turn on and off in complex temporal patterns, and/or may have two or more alternative steady activity states, e.g., levels of expression. It is convenient to designate these genes and products, whose activities can vary within and especially between attractors, as “green”. For example, genes having an expression level that varies between different cell types of the organism may be designated as green.

The genes within any one green island can form simple or complex regulatory sub-networks of the entire genetic network. For example, the expression levels of genes within a given subnetwork, e.g. green island, may exhibit one or more attractors, e.g., recurrent states of expression level. Additionally, expression levels of genes within a given green island may exhibit correlated behavior. However, the expression levels of genes and gene products within different green islands are functionally isolated from one another in the sense that alterations in the expression levels or activities of variables, or genes, gene products in one island cannot perturb the expression levels of genes or activities of variables or genes within other isolated green islands. That is, variables within different green islands exhibit generally uncorrelated behavior.

In the ordered regime, nearby states in state space tend, on average, to lie on trajectories that converge closer to one another in state space. That is, if homeostasis is defined as “return after perturbation”, in the ordered regime, the dynamics are homeostatic. Specific algorithmic measures are known in the art, such as the “Derrida Curve” to characterize whether the dynamics of a model or real system show convergent flow in state space. See, The Origins of Order, incorporated herein by reference. Briefly, the two copies of the system are “initiated” at pairs of states at different initial “distances”. For binary networks, a convenient measure of the distance between two binary states is the fraction of binary variables by which they differ, called the normalized Hamming distance, H. Thus, for example, (1111111111) and (0111111110) overlap in 8 of ten positions and differ by 2 so that the normalized Hamming distance is 0.2. For continuous variables, a generalized continuous euclidian metric is convenient to measure the distance between to continuous vectors of gene and product activities.

In numerical studies described in Origins of Order, each copy of the network is allowed to undergo a short time evolution, corresponding to a single state transition in a synchronous Boolean network, or a short interval corresponding to the time scale for genes to change activations states or to turn on and off in that organism. The result of this time evolution is that each copy arrives at a successor state from its initial state. The distances between the successor states are measured. The final distance between states or D(T+1) may be less than, equal to, or greater than the initial distance, D(T) between the states. In the ordered regime, states at all initial distances, on average, tend to lie on trajectories that converge. This is revealed by the fact that, for such systems, for all pairs of initial states, at different initial distances, on average, D(T+1) is less than D(T).

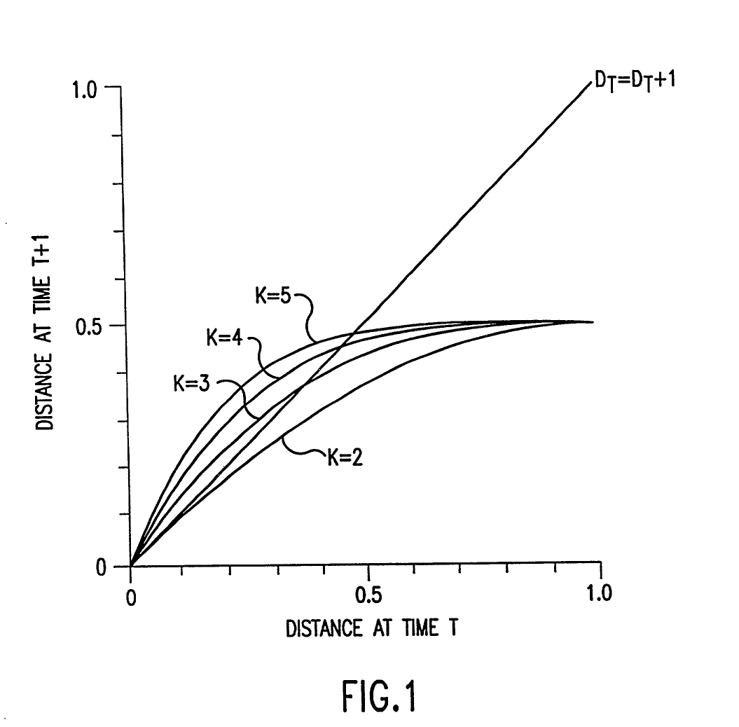

FIG. 1 shows a Cartesian coordinate system with D(t) plotted on the X axis, and D(T+1) plotted on the Y axis, the resulting “Derrida curve” averaged for many pairs of initial states at each initial distance, characterizes whether the system is in the ordered regime or the chaotic regime. The Derrida curve is a recurrence relation showing the expected distance Dt+1 between two states at time T+1 as a function of the normalized distance, Dt between two states a moment earlier. The main diagonal, Dt+Dt+1 shows the conditions under which two initial states lie on trajectories that neither diverge nor converge in state space. For values of K, the number of inputs per gene, greater than 2, the Derrida curve lies above the main diagonal for small initial distances between initial states, Dt. This corresponds to the first step in an expanding avalanche of damage and is a signature of chaotic behavior and sensitivity to initial conditions. For K=2 or less, the Derrida curve is below the main diagonal for all initial distances, Dt, corresponding to convergence in state space. K=2 is the phase transition to chaos.

In the ordered regime, the plot is everywhere below the “main diagonal” where D(T+1)=D(t). In the chaotic regime, for some initial distances, typically small initial distances, states tend to diverge. This is “the butterfly effect”, or sensitivity to initial conditions. Here, in the chaotic regime, D(T+L) is greater than D(t) for these initial distances.

A further characteristic of the ordered regime concerns the propagation of “damage” in such networks following perturbation of one or more variables of the network as evidenced by numerical simulations described in Origins of Order by Kauffman, above. Consider two identical copies of a network of variables. Alter or perturb the activity value of a single variable, e.g. expression level of a gene, or product in one of the two network copies. Allow both network copies, the unperturbed, and the perturbed, to evolve forward for a time sufficient to allow at least some of the network variables to change state. Determine the state or activity level of network and compare the state of the perturbed and unperturbed copies. A variable, gene, or product within the perturbed copy may be defined as “damaged” if state or level of activity of the variable is ever different, one or more times, from the state or level of activity of the corresponding variable in the unperturbed copy. The steps of allowing each network to evolve one or more steps and determining and comparing the level of activity of the networks may be carried out repeatedly. Given this definition, a site can be different in its activities from the unperturbed site many times, but it is only damaged once and remains damaged thereafter.

Further, given this definition of damage, one can define the size of an avalanche of damaged variables, genes, products or “sites” following the initial perturbation to a single gene, gene product, or site. Generically, for a network in the ordered regime, the size distribution of damage avalanches is a power law distribution with many small avalanches and few large ones, due to many random choices of which gene at which network state to perturb. To illustrate the power law, the logarithm of the size of the avalanche is plotted on a X axis and the number of avalanches at that size is plotted on a Y axis of a Cartesian coordinate system. A power law produces a straight line with a negative slope.

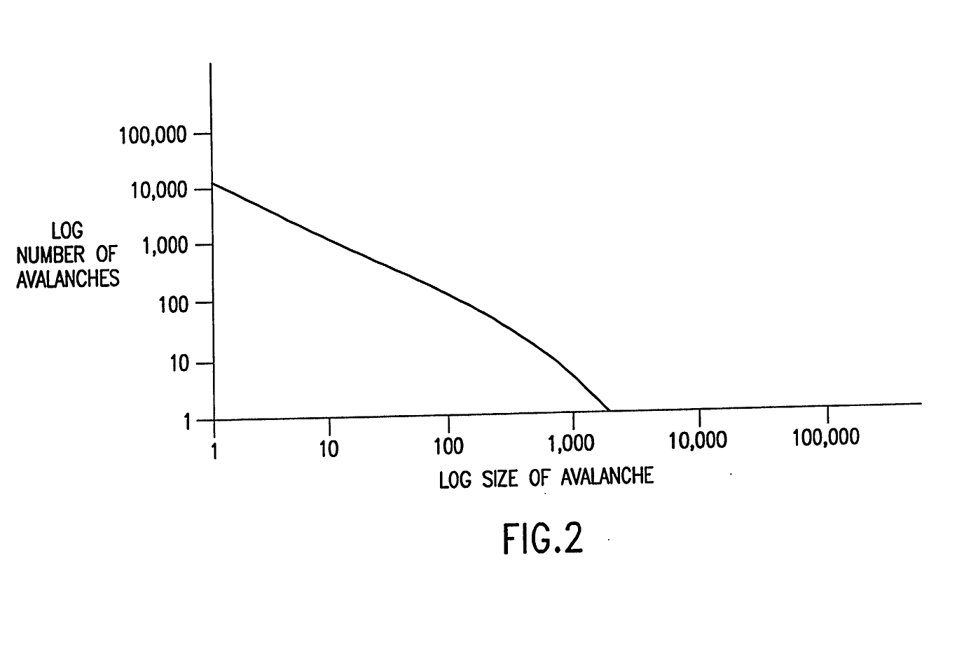

FIG. 2 shows that a simulated binary network in the ordered regime has a power law distribution of avalanches of changes in gene activities produced by reversing the activation state of a single randomly chosen gene. The simulated network had N=65,000 on or off genes, which is about equal to the number of genes in a human cell. The distribution shows a finite cutoff with maximal avalanche size about equal to 2 or 3 times the square root of the number of genes in the system. Thus, in the ordered regime very near the phase transition to chaos, the power law size distribution has a maximum size avalanche that scales as a rough square root function of the number of genes. Deeper in the ordered regime, as measured, for example, by the derrida curve lying further below the main diagonal, the distribution of avalanches is a similar, slightly steeper power law, with a smaller maximum size avalanche.

In the chaotic regime, the size distribution of avalanches shows a similar power law distribution of relatively small avalanches, and a “spike” of huge avalanches that may involve between 20% to 50% or more of the genes, the center of the spike shifting to higher fractions as the network is deeper in the chaotic regime as measured by the how much of the derrida curve lies above the main diagonal.

In the chaotic regime, rather than there being isolated green islands of genes and products whose activities can vary within, or more importantly between attractors, there is instead a vast percolating “green sea” of connected genes and products all of which can vary in activity within or between attractors. In the chaotic regime there may be one or more isolated red frozen islands.

In the ordered regime, if damage is initiated by stimulating, inhibiting or otherwise perturbing a gene or gene product in an isolated green island, the propagating avalanche of damage is entirely, or almost entirely confined to that green island. This reflects the fact that alterations cannot propagate across the fixed percolating red frozen structure that isolates the green islands from one another. In contrast, in the chaotic regime, perturbation of an initial gene or product may unleash an avalanche that spreads to a finite fraction of the other genes or products, corresponding to the huge avalanches seen in the chaotic regime. Here, “finite” means that the size of the largest avalanche scales linearly with the size of the network.

The size distribution of isolated green islands themselves is a power law, with more small islands than large ones. The average size of an isolated green island scales logarithmically with the size of the network.

In the ordered regime, a fundamental feature of the dynamics of the simulated networks is that when the network settles to an attractor, each green isolated island is itself, as a sub-network of the entire network, on an attractor. Each isolated green island may be capable of one or more different alternative attractors. For example, in the piecewise linear case deep in the ordered regime, these different attractors correspond to different steady state expression levels of the genes and their products. Thus, importantly, the set of all the different attractors of the entire network correspond to the“frozen red sea” in the same fixed state on all attractors, and the green islands in their different attractors. Thus, the behavior of the whole network in the ordered regime is fundamentally “combinatorial”. For example, let a network have three isolated green islands, A, B, and C. Let A have two alternative attractors, B have three alternative attractors, and C have four alternative attractors, perhaps, for example, each attractor corresponds to a different steady state level of activities of genes and products in the corresponding green isolated island. Then the total number of attractors of the entire network is the product of the number of alternatives in the three isolated green islands, 2×3×4=24. The alternative “choices” made by the different attractors constitute a kind of “epigenetic code” that specifies the attractor of the entire network. Given the identification of an attractor of the entire network and a cell type of an organism, this epigenetic code word then specifies the cell type in question. See, Origins of Order incorporated herein by reference.

The behavior of binary networks, piecewise linear networks, and sigmoidal networks are similar, except that the latter two continuous networks tend to exhibit “green islands” in which genes and products are at different steady state levels of activities on the different attractors of each of those islands, whereas in the binary synchronous case, the activation states of genes within one island generally decrease or increase in complex patterns on the state cycle attractors of each island.

Numerical investigation of Boolean and piecewise linear networks have revealed the homologous ordered and chaotic regimes and the same scaling law for the phase transition between order and chaos as two parameters, P, characterizing a specific bias in the response function, and the number of inputs per variable or gene, K, are tuned. See, Origins of Order, and Glass and Hill 1998, incorporated herein by reference. More recently, Hill and colleagues have shown the same phase transition between order and chaos in two parameter plane corresponding to biases towards canalizing functions on one axis and the number of inputs per gene, K, on the other axis. A canalizing function may be defined as any Boolean function having the property that it has at least one input having at least one value (1 or 0) which suffices to guarantee that the output of a variable or element regulated by the function assumes a specific value (1 or 0). See Origins of Order, incorporated herein by reference, for a more complete discussion of canalizing functions. A logical “and” is such a function because if either the first or second input is 0, the regulated output is guaranteed to be 0. By contrast, a logical “exclusive or” is not a canalizing function because not single state of either input guarantees that the behavior of the output.

Recent results very strongly suggest that eukaryotic genes are biased to regulation by canalizing functions. This is based actual observed transcription regulation for eukaryotic genes with K=3,4, and 5 known direct regulatory inputs.

Mathematical analysis of model genetic networks with the observed biases towards the same distribution of high numbers of canalizing functions as seen in real regulated eukaryotic genes firmly indicates by derrida curves, the presence of percolating red frozen structures and power law distributions of damage avalanches, that real eukaryotic cells lie in the ordered regime. In short, mathematical and numerical work known in the art suggests the same broad behavior regimes in binary and piecewise linear, and, with less data, sigmoidal, model genetic networks.

Thus, it is very likely that real cells, eukaryotic and prokarytic, lie in the ordered regime with isolated green islands whose alternative attractors are central to cell differentiation and may constitute an epigenetic code.

5.2 Measurement of Gene Activity

Wherein, the term activation state of a gene refers to the level of gene activity, i.e. the level of expression of the gene. The products of gene expression are transcripts (e.g. hnRNA or mRNA) and translation products (i.e. proteins). Thus, the level of expression of a gene in a biological sample can be characterized, e.g. measured, qualitatively or quantitatively, or both, by detecting the abundance of transcripts or protein products of that gene present in the biological sample.

The cell, set of cells, or tissue sample may correspond to cells or tissue obtained in vivo from an organism or to cells or tissue obtained or grown in vitro.

In general, the methods of the invention comprise measuring the activation state or level of expression of one or more genes within one or more biological samples.

Methods for measuring transcripts (e.g. hnRNA or mRNA) and protein products are well known in the art. For example, and not by way of limitation, a parallel method for measuring the level of gene activity within a sample includes the use of nucleotide arrays such as those which are commercially available from Affymetrix Incorporated, as described in U.S. Pat. No. 5,837,832, which is hereby incorporated herein by reference in its entirety. Using such arrays, transcript expression from a plurality of genes can be detected and quantified in parallel over a wide range of expression levels to allow comparison of expression levels for a plurality of different genes within a biological sample. The relative expression of many genes can be simultaneously determined. Changes in the level of expression over time or between samples may also be measured qualitatively or quantitatively or both.

In an alternative approach, SAGE analysis (serial analysis of gene expression) or similar analyses may also be used to characterize the activation state of a gene. SAGE, as described in U.S. Pat. No. 5,695,937, incorporated herein by reference in its entirety, provides a method for the rapid analysis of numerous transcripts in order to identify the overall pattern of gene expression in different cell types or in the same cell type under different physiologic, developmental or disease conditions. The method is based on the identification of a short nucleotide sequence tag at a defined position in a messenger RNA. The tag is used to identify the corresponding transcript and gene from which it was transcribed. By utilizing dimerized tags, termed a “ditag”, SAGE allows elimination of certain types of bias which might occur during cloning and/or amplification and possibly during data evaluation. Concatenation of these short nucleotide sequence tags allows the efficient analysis of transcripts in a serial manner by sequencing multiple tags on a single DNA molecule, for example, a DNA molecule inserted in a vector or in a single clone. Each technique for characterizing the level of gene transcription activity analyzes the RNA or hnRNA, or mRNA content of a single cell, a set of cells of the same cell type, or a tissue sample which may have one or more cell types to reveal the relative abundances of transcripts of thousands of different genes simultaneously.

The level of gene expression may also be measured by characterizing the abundance of translation products (e.g. proteins) within a biological sample. For example, and not a limiting example, the abundance of proteins within a biological sample may be characterized by using two dimensional protein gels or similar parallel analysis methods, including, but not limited to, analyses of translation rates and phosphorylation states.

In all embodiments of the invention, the characterization (e.g. qualitative and/or quantitative measurement) of gene expression may be performed at a single point in time, or at plurality of succeeding points in time to establish a temporal record or time series of the expression level of a plurality of genes within a biological sample. Thus, in a non-limiting example, a time series might correspond to a series of measurements of the expression level of genes within a cell line following introduction of a hormonal or other stimulus. For example, a hormonal stimulus may be introduced to induce differentiation of the cell line. A corresponding time series of measurements of gene expression could be acquired from a similar cell line not receiving the hormonal or other stimulus and can be compared to the corresponding time points in the treated sample. In another example, the level of gene expression in a population of cells undergoing a cell cycle may be measured repeatedly to acquire a time series.

In certain cases, it is now possible to measure and quantify the expression level of genes from single cells, such as neurons. Additionally, using methods described in the U.S. patent application No. 09/165,794 filed Oct. 2, 1998, by inventors Kauffman and Ballivet, hereby incorporated by reference in its entirety, the bound and unbound states of one or a plurality of cis acting sites in a single cell or set of cells may be assayed to establish in parallel the “cis activity” state of a cell or cells.

5.3 Perturbation of Physiological State of the Biological Sample

In accordance with the invention, the physiological state of a biological sample may be perturbed by modulating the expression of one or more target genes, or the activity of one or more target gene products, in the sample. Such modulation includes inhibiting or enhancing the expression of the gene or the activity of the gene product, using methods well known in the art.

5.3.1 Methods of Inhibiting Expression Of A Target Gene Or Activity Of A Target Gene Product

Methods of inhibiting gene expression include, but are not limited to, knocking out the gene expression by mutating the target gene such that a functional gene product is not produced, and inhibiting the gene expression by adding a compound that inhibits the expression of the gene. Such inhibitory compounds include, but are not limited to, anti-sense mRNA, ribozymes, and triple helix forming oligonucleotides. In addition, a compound that down-regulates gene expression, such as a metabolite that binds an apo-repressor to form activated repressor, can be used to inhibit gene expression. Moreover, compounds that inhibit the activity of the protein product of a target gene can be used to inhibit the effect of that protein on a downstream target site (e.g., nucleotide regulatory region or another protein), and thereby perturb the physiological state of the biological sample. For example, in specific embodiments for inhibiting the activity of a receptor protein, antibodies or other ligand analogues that bind the receptor and inhibit the ability of the natural ligand to bind the receptor may be added to the biological sample.

5.3.1.1 Perturbation Through Targeted Inhibition Of Gene Expression

Among the compounds which may perturb the physiological state of a biological sample through inhibition of the expression of a particular gene are antisense, ribozyme, and triple helix molecules. Techniques for the production and use of such molecules are well known to those of skill in the art.

Antisense RNA and DNA molecules act to directly block the translation of mRNA y hybridizing to targeted mRNA and preventing protein translation.

Antisense approaches involve the design of oligonucleotides (either DNA or RNA) that are complementary to target gene mRNA. The antisense oligonucleotides will bind to the complementary target gene mRNA transcripts and prevent translation. Absolute complementarity, although preferred, is not required. A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have recently shown to be effective at inhibiting translation of mRNAs as well. See generally, Wagner, R., 1994, Nature 372:333-335. Thus, oligonucleotides complementary to either the 5′- or 3′-non-translated, non-coding regions of the target gene could be used in an antisense approach to inhibit translation of endogenous target gene mRNA. Oligonucleotides complementary to the 5′ untranslated region of the mRNA should include the complement of the AUG start codon. Antisense oligonucleotides complementary to mRNA coding regions are less efficient inhibitors of translatisAon but could be used in accordance with the invention. Whether designed to hybridize to the 5′-, 3′- or coding region of target gene mRNA, antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

Regardless of the choice of target sequence, in vitro studies can first be performed to quantitate the ability of the antisense oligonucleotide to inhibit gene expression. These studies may utilize controls that distinguish between antisense gene inhibition and nonspecific biological effects of oligonucleotides. These studies may also compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. W088/09810, published Dec. 15, 1988) or the blood-brain barrier (see, e.g., PCT Publication No. W089/10134, published Apr. 25, 1988), hybridization-triggered cleavage agents. (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5 -methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an -anomeric oligonucleotide. An -anomeric oligonucleotide forms specific double-stranded hybrids with complementary RNA in which, contrary to the usual -units, the strands run parallel to each other (Gautier et al., 1987, Nucl. Acids Res. 15:6625-6641). The oligonucleotide is a 2-O-methylribonucleotide (Inoue et al., 1987, Nucl. Acids Res. 15:6131-6148), or a chimeric RNA-DNA analogue (Inoue et al., 1987, FEBS Lett. 215:327-330).

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

While antisense nucleotides complementary to the target gene coding region sequence could be used, those complementary to the transcribed untranslated region are most preferred.

A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304-310), the promoter contained in the 3 long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), etc. Any type of plasmid, cosmid, YAC or viral vector can be used to prepare the recombinant DNA construct which can be introduced directly into the tissue site; e.g., atherosclerotic vascular tissue. Alternatively, viral vectors can be used which selectively infect the desired tissue, in which case administration may be accomplished by another route (e.g., systemically).

Ribozymes are enzymatic RNA molecules capable of catalyzing the specific cleavage of RNA. The mechanism of ribozyme action involves sequence specific hybridization of the ribozyme molecule to complementary target RNA, followed by an endonucleolytic cleavage. Ribozyme molecules designed to catalytically cleave target gene mRNA transcripts can also be used to prevent translation of target gene mRNA and expression of target gene. (See, e.g., PCT International Publication WO90/11364, published Oct. 4, 1990; Sarver et al., 1990, Science 247:1222-1225). While ribozymes that cleave mRNA at site specific recognition sequences can be used to destroy target gene mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591. For example, there are hundreds of potential hammerhead ribozyme cleavage sites within the nucleotide sequence of rchd534 and fchd540 cDNA. Preferably the ribozyme is engineered so that the cleavage recognition site is located near the 5′end of the target mRNA; i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO 88/04300 by University Patents Inc.; Been and Cech, 1986, Cell, 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in target gene.

As in the antisense approach, the ribozymes can be composed of modified oligonucleotides (e.g. for improved stability, targeting, etc.) and should be delivered to cells which express the target gene in vivo, e.g., endothelial cells. A preferred method of delivery involves using a DNA construct “encoding” the ribozyme under the control of a strong constitutive pol III or pol II promoter, so that transfected cells will produce sufficient quantities of the ribozyme to destroy endogenous target gene messages and inhibit translation. Because ribozymes, unlike antisense molecules, are catalytic, a lower intracellular concentration is required for efficiency.

Nucleic acid molecules to be used in triple helix formation for the inhibition of transcription should be single stranded and composed of deoxyribonucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC+ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC paris, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′,3′-5′manner, such that they base pair with first one strand of a duplex and then the other, eliminating the necessity for a sizeable stretch of either purines or pyrimidines to be present on one strand of a duplex.

Target gene expression can also be reduced by inactivating or “knocking out” the target gene or its promoter using targeted homologous recombination. (E.g., see Smithies et al., 1985, Nature 317:230-234; Thomas & Capecchi, 1987, Cell 51:503-512; Thompson et al., 1989 Cell 5:313-321; each of which is incorporated by reference herein in its entirety). For example, a mutant, non-functional target (or a completely unrelated DNA sequence) flanked by DNA homologous to the endogenous target gene (either the coding regions or regulatory regions of the target gene) can be used, with or without a selectable marker and/or a negative selectable marker, to transfect cells that express target in vivo. Insertion of the DNA construct, via targeted homologous recombination, results in inactivation of the target gene.

Alternatively, endogenous target gene expression can be reduced by targeting deoxyribonucleotide sequences complementary to the regulatory region of the target gene (i.e., the target promoter and/or enhancers) to form triple helical structures that prevent transcription of the target gene in target cells in the body. (See generally, Helene, C. 1991, Anticancer Drug Des., 6(6):569-84; Helene, C., et al., 1992, Ann, N.Y. Accad. Sci., 660:27-36; and Maher, L. J., 1992, Bioassays 14(12):807-15).

5.3.1.2 Disruption of Target Genes

Endogenous target gene expression can also be reduced by inactivating or “knocking out” the target gene or its promoter using targeted homologous recombination. (E.g., see Smithies et al., 1985, Nature 317:230-234; Thomas & Capecchi, 1987, Cell 51:503-512; Thompson et al., 1989 Cell 5:313-321; each of which is incorporated by reference herein in its entirety). For example, a mutant, non-functional target (or a completely unrelated DNA sequence) flanked by DNA homologous to the endogenous target gene (either the coding regions or regulatory regions of the target gene) can be used, with or without a selectable marker and/or a negative selectable marker, to transfect cells that express target in vivo. Insertion of the DNA construct, via targeted homologous recombination, results in inactivation of the target gene. Such approaches can be adapted for use in humans provided the recombinant DNA constructs are directly administered or targeted to the required site in vivo using appropriate viral vectors, e.g., vectors for delivery vascular tissue.

An example of such an animal model is the apo-deficient mouse, which is an animal model for astherosclerosis, in which the ap-E gene has been disrupted (Plump et al., 1992, Cell 701:343-353). Using the methods disclosed herein, biological samples from animal modesl such as the apo-deficient mouse can be analyzed to define green islands correlated with an atherosclerotic disease state.

5.3.2 Perturbation Through Targeted Gene Expression

The physiological state of the cell can be perturbed by selectively regulating the expression of one or more genes. For example, a given gene can be genetically engineered so that its expression is controlled by a specialized promoter. Host cells can be transformed with the target gene controlled by appropriate expression control elements (e.g., promoter, enhancer, sequences, transcription terminators, polyadenylation sites, etc.), and a selectable marker. Following the introduction of the recombinant DNA construct, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the target gene in a controlled manner. Using these methods, the target gene can be engineered to be expressed under the control of a highly expressed promoter, for example. Such promoters may be constitutive, or regulatable such that high levels of expression can be induced upon addition of an inducing compound or other stimulus (e.g., temperature shift in the case of a temperature sensitive promoter). Alternatively, gene which is normally highly expressed in a given cell type and/or under certain physiological conditions can be selectively down-regulated by adding a factor known to negatively regulate the recombinant promoter.

A number of selection systems may be used for introduction of the recombinant construct, including but not limited to the herpes simplex virus thymidine kinase (Wigler, et al., 1977, Cell 11:223), hypoxanthine-guanine phosphoribosyltransferase (Szybalski & Szybalski, 1962, Proc. Natl. Acad. Sci. USA 48:2026), and adenine phosphoribosyltransferase (Lowy, et al., 1980, Cell 22:817) genes can be employed in tk-, hgprt- or aprt-cells, respectively. Also, antimetabolite resistance can be used as the basis of selection for dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147) genes.

5.4 Characterization of Perturbed of Genetic Networks

Another embodiment of the present invention utilizes experimental perturbations or stimuli to modify the expression level of a predetermined gene, followed by characterization of the subsequent expression levels of a plurality of genes in the network to identify genes within the same green island. Damage, e.g. subsequent changes in the expression levels of one or more genes in a network having a gene which has been perturbed as compared to the expression levels of corresponding genes in an unperturbed network, will be substantially confined to genes in the same green island. A gene, or product within the perturbed copy may be defined as “damaged” if the expression level of the gene is ever different, one or more times, from the state or level of activity of the corresponding variable in the unperturbed copy. Given this definition, a site can be different in its activities from the unperturbed site many times, but it is only damaged once and remains damaged thereafter.

Thus, by characterizing the expression levels of genes within a biological sample that has not been perturbed and characterizing the expression levels of genes within a biological sample that has been perturbed and comparing the expression levels of corresponding genes in the perturbed and unperturbed sample one can identify which genes in the sample belong to the same green island.

Perturbations can be achieved by a variety of means known in the art as described in Sections 5.3 above and 5.6, below, including cloning an exogenous promoter or enhancer upstream from the gene in question, and increasing the activity of the gene via that promoter. RNA chip, protein gel etc analysis is then performed to determine which other genes or products change their activities following the perturbation. In addition to cloning upstream cis sites, injection of complementary RNA which hybridizes to the mRNA of specific gene, antisense, phage display peptides that bind the RNA in question or modulate the activity of cis sites, extra copies of cis sites injected into cells, small molecules that modulate the activity of the gene or product in question, or any other perturbation can be used to perturb one or more genes in a genetic network and to initiate avalanches of change within the network.

As a non-limiting example, a preferred embodiment of the invention proceeds by characterizing the expression level of genes within a biological sample, altering or perturbing the expression level of a single predetermined gene within the biological sample; allowing the perturbed biological sample and an otherwise identical unperturbed biological sample to evolve for a time sufficient to allow the expression levels of at least some genes to change; characterizing the expression level of a plurality of genes within the perturbed and unperturbed biological samples; and comparing the expression levels of corresponding genes within the perturbed and unperturbed samples to identify genes that experience a change of expression level or state in response to the perturbation.

The steps of altering or perturbing, allowing each sample to evolve in time, characterizing, and comparing the expression levels of genes within the samples may be repeated.

Characterization of the expression level of genes in the sample or the abundance of proteins in the sample may be carried out using any methods for characterizing the expression level of genes or proteins including but not limited to nucleotide arrays, SAGE, or two dimensional protein gels, as described above.

It is possible that the network may exhibit more than one green island. In such cases, the presence of more than one green island may be ascertained and genes belonging to each green island identified.

5.5 Measures of Mutual Information

If there is correlation between the activation states (i.e. expression levels) of a number of genes, then the current or past expression level of one gene may directly or indirectly influence the current or future expression level of one or more of the remaining genes. For example, if a protein expressed by a first gene inhibits the transcription or translation of a second gene then activation of the first gene likely reduces the level of expression of the second gene. Thus, the activation state of the second gene is correlated with the activation state of the first gene. Given this definition of correlation between genes, if the activation states of two or more genes are correlated and the regulatory rules corresponding to the genes are known, then knowledge of the state of one of the genes provides information regarding the past, current, or future states of the remaining genes. The activities of genes within a given green island are generally correlated whereas the activities of genes in different green islands are generally uncorrelated. Thus, genes identified as having correlated activities or levels of expression generally belong to the same green island whereas genes identified as having uncorrelated activities or levels of expression generally belong to different green islands.

In general, however, the regulatory rules corresponding to the genes may not be known. One embodiment of the present invention, to characterize which genes are in the same green island, is based on methods to measure correlations between changes in the expression level of genes within one island, and the lack of correlation between changes of expression levels of genes within different islands. The regulatory rules corresponding to the expression of the genes need not be known to characterize the correlation between the expression levels of the genes. Without limitation, one such measure of the correlation between the expression levels of any two genes is given by mutual information. The mutual information, M, between the expression levels of any two genes, A and B, may be described by

$${\sum }_{i = 1}^{m} p ( Ai ) log ( p ( Ai ) ) + {\sum }_{k = 1}^{n} p ( Bk ) log ( p ( Bk ) ) + {\sum }_{i = 1}^{m} {\sum }_{k = 1}^{n} p ( AiBk ) log ( p ( AiBk ) )$$

Where p(Ai) is the probability that the expression level of gene A is in the ith state or level of activity, p(Bj) is the probability that the expression level of gene B is in the jth state or activity level, and p(Ai,Bj) is the joint probability that the expression level of gene A is in the ith state while the expression level of gene B is in the jth state. The sum of terms (Ai)logp(Ai) represents the entropy of the gene A and is evaluated over the m discriminable expression levels exhibited by gene A. The sum of terms p(Bj)logp(Bj) represents the entropy of gene B and is evaluated over the n discriminable expression levels exhibited by gene B. The sum of terms p(AiBj)logp(AiBj) represents the joint entropy of genes A and B. The joint entropy is evaluated over the m discriminable expression levels states of gene A and the n discriminable expression levels of gene B. Thus, the mutual information of the two genes is the sum of the entropy of gene A, H(A)=p(Ai)logp(Ai), plus the entropy of gene B, H(B)=p(Bj)logp(Bj) minus the joint entropy, H(AB) p(AiBj)logp(AiBj). That is, M=H(A)+H(B)−H(AB).

If the probabilities p(Ai), p(Bj), and p(AiBj) are not known, the probability that a gene exhibits a given expression level may be replaced by the fraction of time the gene exhibits a given expression level. For example, in one embodiment of the present invention, the genes A and B correspond to a pair of genes in a given cell type or in different cell types. As an example, consider a set of genes modeled as a synchronous Boolean network on a single state cycle with 10 states within the state cycle and wherein the activity of each gene may be described by one of two states. The state of the expression level of each gene may be defined as either on (e.g. active) or off (e.g. inactive). In this case, for example, the entropy of A is given by the sum of p(Ai)logp(Ai), where the term p(Ai)logp(Ai) is evaluated over two cases: first where p(Al) is the fraction of time that gene A is “on” and, second, where p(A2) is the fraction of time that gene A is off. The entropy of B is evaluated similarly based on the fraction of time B is either on or off. The joint entropy considers the fraction of time that genes A and B are simultaneously on, the fraction of time that genes A and B are simultaneously off, and the fraction of time that A is on when B is off and the fraction of time that A is off when B is on.

The mutual information between any two variables is non-zero only when both variables exhibit correlated unfixed changes in state. For example, when the expression levels of genes A and B are uncorrelated, then H(AB)=H(A)+H(B) and the mutual information is 0. Similarly, when the expression level of one of two genes is fixed, then its entropy is 0, and the joint entropy is equal to the entropy of the remaining, unfixed gene. Thus, the mutual information between two genes is also 0 when the expression level of at least one of the two genes is fixed.

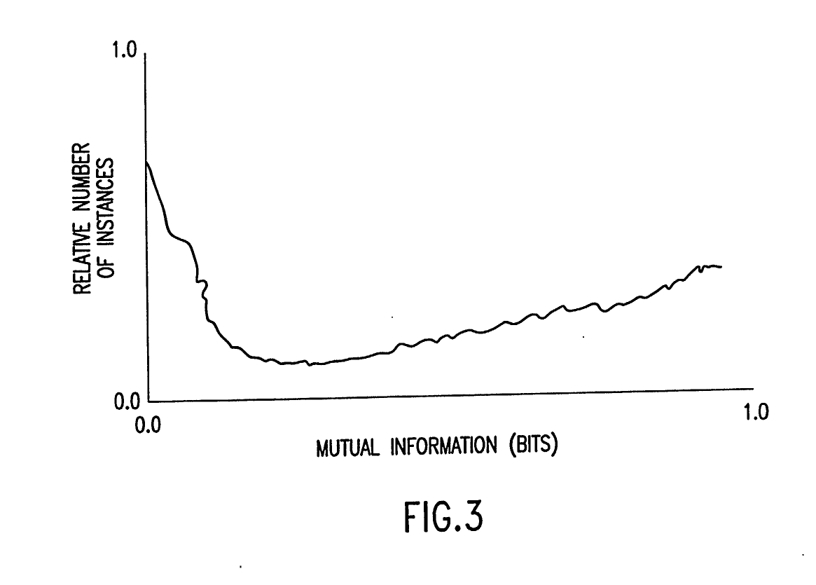

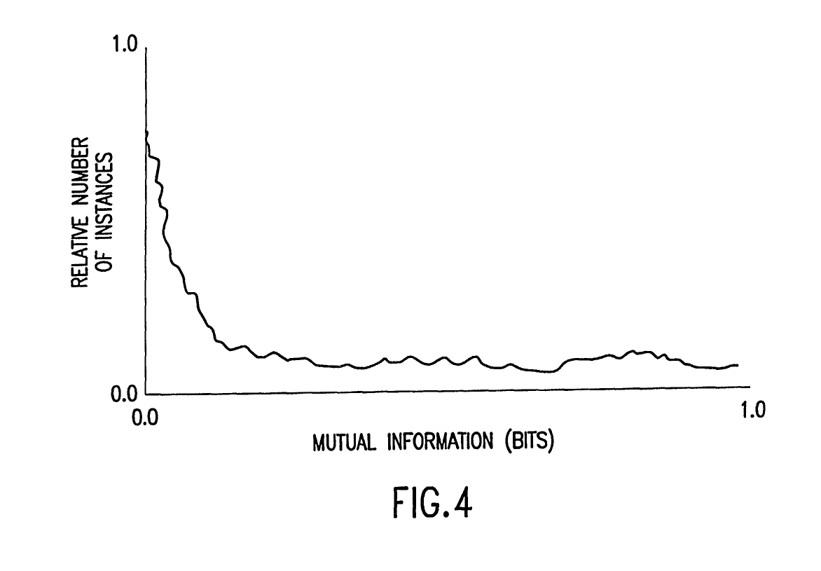

For example, one can test whether two genes, A and B, on one attractor of a Boolean network are in the same isolated green island, or in different islands. To do so, a mutual information test is carried out between the expression levels of all pairs of genes whose activities change within or between attractors (or within or between cell types of real organisms). Numerical analysis using synchronous Boolean networks in the ordered regime generated by matching the known distribution of canalizing functions observed in regulated eukaryotic genes, shows that this test suffices to distinguish most genes within the same green island from genes in different green islands, FIG. 3. Similarly analysis of mutual information between the genes but where the analysis is taken over many or all the alternative attractors of the same network, again shows that this measure suffices to distinguish most genes that are in the same green island from those that are in different islands, FIG. 4.

In one embodiment of the present invention, the corresponding characterization of the expression levels of genes within one or more cells, sets of cells or tissue samples includes characterization of the expression level of genes within one or more biological samples such as one or more cells, sets of cells or tissues. If a gene (or protein abundance) exhibits a range of expression levels, defining or binning the observed abundances of each gene's transcription activity into two or more discriminable ranges allows mutual information measures to be constructed. Then, mutual information tests are carried out between the expression levels of all pairs of genes (or protein abundances) whose activities change within or between cells, cell types or tissue samples to identify genes that substantially correspond to members of the same or different green islands. Mutual information tests may also be carried out between pairs of proteins whose levels or concentrations change between cells, cell types, or tissue samples.

In another embodiment of the present invention, characterization of the expression levels of genes (or protein abundances) within a one or more biological samples is repeated at least once to establish a temporal record of the expression levels of the genes or proteins states. Then, mutual information tests are carried out between all pairs of genes (or protein abundances) whose activities change over the temporal record to identify genes that are members of the same green island.

5.6 Characterization of Patterns of Gene Expression Levels

Another embodiment of the invention is based on the analysis of the level of gene expression from more than one alternative cell type of the same organism. As a non-limiting example of the algorithm, consider the hypothetical case of a genetic network with three isolated green islands, A, B and C, where A has two alternative steady state attractors, B has three alternative steady state attractors, and C has four alternative steady state attractors. The attractors within each island represent substantially recurrent patterns of the expression levels of genes within each island that generally occupy a sub-volume of all the space containing all possible states of the expression levels of genes within each island. The genes within each island can occupy one or more discriminable levels of expression and each state or level of expression of a gene corresponds to one of the attractors exhibited by the island containing the gene.

By way of non-limiting example, assume that A contains 5 genes, B contains 11 genes, and C contains 21 genes. In this example, it is further assumed without limitation, that each expression level of each of the 37 genes can be characterized and/or discriminated using the measurement approaches described above.

Consider the total set of 5+11+21=37 genes associated with the green islands. The total number of alternative attractors associated with the 37 variables of the network is given by the product of the number of attractors of the three islands, or 2×3×4=24. Thus, the network as a whole exhibits 24 attractors, which correspond to a recurrent pattern of expression levels of the 37 network genes. As a comparison, Hydra has about 13 cell types, e.g. attractors, and humans have about 265 cell types, e.g. attractors.

For reference purposes, each gene may be assigned a number from 1 to 37. However, the number of a given gene is not a function of the island to which the gene belongs. Thus, a given gene may belong to any of the three islands.

A preferred embodiment of the invention comprises of randomly specifying an initial ordering of the 37 genes associated with the 3 islands from among the 37! possible orderings of the genes. Without loss of generality, let the initial ordering be the natural, numerical ordering 1,2,3, . . . , 37. Beginning with gene 1, the 37 genes may be grouped into 37 successively larger sets of genes containing from 1 to 37 genes. For example, set 1 might include only gene 1, set 2 might include genes 1 and 2, set 3 might include genes 1-3, and set 37 might include genes 1-37.

Each set of genes has a given number of patterns wherein each pattern corresponds to a possible combination of the expression levels associated with the genes contained in the set. The algorithm proceeds by identifying the number of patterns of expression levels associated with each set of genes. If gene 1 happens to be associated with island C, which has four attractors, then, according to this example, variable 1 will exhibit four discriminably different activity levels, e.g. patterns.

Next, the number of different patterns of activity associated with the set of genes 1 and 2 are identified. By way of example only, assume that gene 2 happens to be in isolated green island A, which has two alternative attractors. Further assume that gene 2 has two discriminable activity levels associated with the two attractors. Then, because genes 1 and 2 belong to different islands the total number of patterns exhibited by genes 1 and 2 will be 8 because the activity of gene 2 may exhibit either of two levels for each of the 4 activity levels of gene 1.

Next, assume that gene 3 lies in green island C. Then, because genes 1 and 3 belong to the same island, the total number of patterns for a set containing genes 1,2, and 3 will remain 8, corresponding to the four attractors of island C, in which genes 1 and 3 reside and exhibit a total of four patterns, corresponding to the steady state levels of both genes 1 and 3 on the four different attractors, times the two patterns due to gene 2 in isolated island A with its two alternative attractors.

Next, assume that gene 4 lies in isolated island B, an island with three attractors. Consequently, the activity level of gene 4 is substantially uncorrelated with the activity levels of genes 1-3. Thus, the set containing genes 1-4 will exhibit 24 total patterns because gene 4 may exhibit one of three activity levels for each of the eight patterns produced by genes 1-3.

Subsequently, repeating the analysis for sets containing genes 1-5, 1-6, . . . , 1-37 will reveal no new patterns of gene activity.

Completing the algorithm for only one ordering among the 37! possible orderings of the genes indicates that there are three isolated islands, one with two attractors, one with three attractors, and one with four attractors. Further, the result indicates that we have data for each of the 37 genes that it alone exhibits 2, 3, or 4 patterns.

Thus, at the end of this single 37 gene analysis, we know for each gene which island it is in, and the number of attractors of that island.

For further confirmation, the algorithm proceeds as follows. First, the 37 genes may be grouped into a different ordering of sets and the analysis repeated. The same spectrum of increases of observed patterns should be observed among the 24 depending upon which order genes from the three islands are sampled. In all cases, a doubling of total patterns, a tripling of total patterns, or a quadrupling of total patterns should be observed.

Further, there may be many islands with the same number of alternative attractors. For example, let C and D be two such islands with four attractors each resulting in a new system with islands A,B,C,D having 2×3×4×4=64 total patterns. The same analysis may be carried out for, say, the natural ordering of genes 1, 2 . . . 37 to reveal which genes are in which island. Each green gene may be classified as to as to how many patterns it alone exhibits. Then a histogram may be produced displaying which genes exhibit 2 patterns, 3 patterns, 4 patterns and so forth. We then pairwise test genes within each such class, say the 2 pattern class, to confirm if jointly they show 2 or 4 total patterns among the 64. This provides a second test to tell if the two genes are in the same island—they jointly exhibit only 2 patterns—or if the two genes are in two islands—they jointly exhibit 4 patterns.

Clearly, the analysis of activity level of genes or proteins associated with the 24 or 64 attractors also reveals the fixed red genes that exhibit a single fixed activity on all attractors.

Meanwhile, given a random ordering among the, here 37 green genes on each of many assays of the X Y analysis of total patterns seen as the ordered set of genes increases, gives an estimate of the number of genes in each class of “island attractors”—the islands with 2 attractors, the islands with 3 attractors, the islands with 4 attractors, etc. This estimate is based on how often a jump in total number of patterns by a doubling, tripling, or quadrupling is found.

If the total number of “green genes” is large, say 20,000 to 40,000 for a human. A similar analysis for a few hundred random orderings among the 20,000 to 40,000 for the first ten to twenty genes in each ordering analyzed across the 265 or so cell types of a human should suffice to characterize most or all of the different green islands in the human genome. More precisely, given hypotheses about the number and size distribution of such islands, the number of ordered sets that must be sampled to ensure that all islands have been sampled at least by one gene in one ordering among the 20,000 to 40,000 green genes can be calculated. From this data, most of the green islands can be recovered at reasonably analysis. Further analysis of which of the remaining 20,000 to 40,000 are in each island can be carried out as above, or by damage analysis or mutual information analysis.

Once a set of genes is shown to be in the same green island, mutual information can also be used to discriminate which genes are direct and which are not direct inputs to one another. For example, if A is an input to B and B is an input to C, but A is not an input to C, then the mutual information about C given A cannot be greater than the amount given by B. The failure of inclusion of data about A to increase mutual information about C establishes that A is not a direct input to C, while the presence of mutual information between A and C, summed over all states of B, establishes that A influences C. Jointly the two indicate an indirect connection between A and C. If mutual information is obtained from a temporal series of characterizations of the activations states of genes or protein states then whether A influences C or C influences A can be discriminated by calculating mutual information for A and C for pairs of times with the state of A before C, versus pairs of times with the state of A after C.

It will be clear to those skilled in the art, that these procedures generalize to cases where the behaviors of “green genes” on each attractor are not steady state behaviors, but have more complex time signatures, so long as any unique and discriminable signature can be assigned to each gene for each of the alternative attractors of the green island of which it is a member. In the simplest case, that signature might be the average over the attractor. Thus, a population of the same cell type distributed randomly around the attractor state cycle or orbit could yield a fine different “average signature” for a given gene for each of the different attractors of the green island in which that gene resides.

In the case of tissues with more than one cell type in the RNA chip snapshot, deconvolution methods based on maximum likelihood estimates are necessary. In these cases, any histological data or other data that gives evidence of the number, fractions, and types of cells in the tissue sample are helpful.

It is possible that cells in an organism have but a single “green” island. Indeed, cells might actually be in the chaotic regime, with a single percolating green sea. If so, the above methods to discover the number of green islands and the number of alternative attractors per island will discover this fact.

Further tests that cells are in the ordered versus chaotic regimes can be based on experimental characterization of derrida curves by analysis of the time patterns of activities of control and perturbed cell populations in which random subsets of 1, 2, or many genes or their products are transiently perturbed. The number of genes perturbed corresponds to the initial distance between the state of the perturbed and unperturbed cell or cell population. This can be affirmed by RNA or protein or both snapshots. The later distance between their states at the RNA or protein levels at short and longer time intervals establishes the derrida curve convergence or divergence for each initial distance and time difference between that pair of states. By averaging over different pairs of states at the same initial distance, the average convergence or divergence in state space can be sampled. This can be achieved by perturbing the same set of genes for different cell types of the same organism taken at different stages of development and pathogenesis.

5.7 Computer Systems

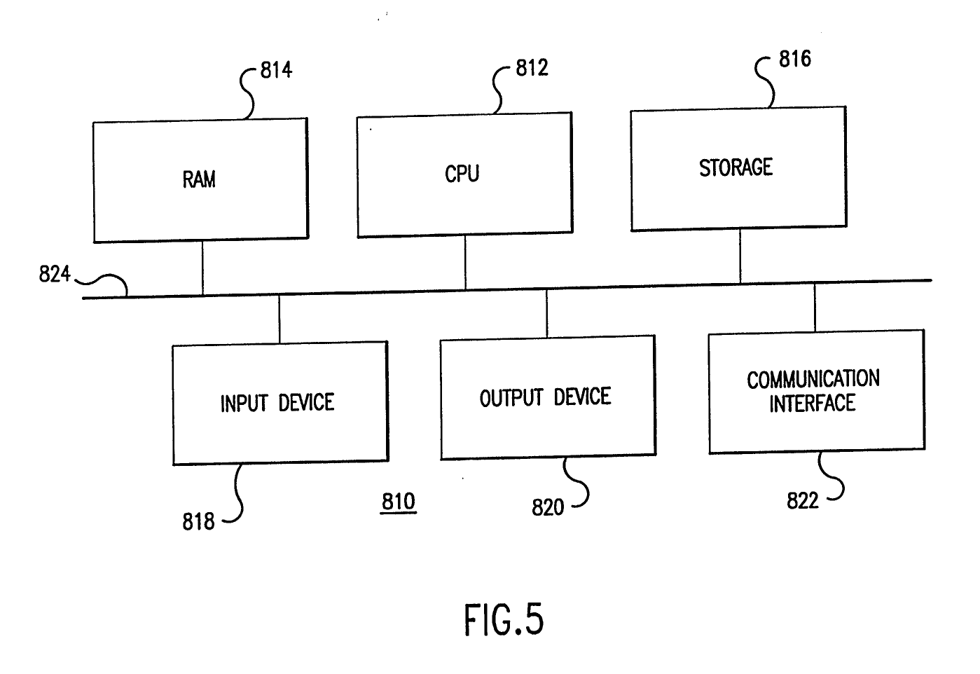

FIG. 5 discloses a representative computer system 810 in conjunction with which the embodiments of the present invention may be implemented. Computer system 810 may be a personal computer, workstation, or a larger system such as a minicomputer. However, one skilled in the art of computer systems will understand that the present invention is not limited to a particular class or model of computer.

As shown in FIG. 5, representative computer system 810 includes a central processing unit (CPU) 812, a memory unit 814, one or more storage devices 816, an input device 818, an output device 820, and communication interface 822. A system bus 824 is provided for communications between these elements. Computer system 810 may additionally function through use of an operating system such as Windows, DOS, or UNIX. However, one skilled in the art of computer systems will understand that the present invention is not limited to a particular operating system.

Storage devices 816 may illustratively include one or more floppy or hard disk drives, CD-ROMs, DVDs, or tapes. Input device 818 comprises a keyboard, mouse, microphone, or other similar device. Output device 820 is a computer monitor or any other known computer output device. Communication interface 822 may be a modem, a network interface, or other connection to external electronic devices, such as a serial or parallel port.

Exemplary configurations of the representative computer system 810 include client-server architectures, parallel computing, distributed computing, the Internet, etc. However, one skilled in the art of computer systems will understand that the present invention is not limited to a particular configuration.

While the above invention has been described with reference to certain preferred embodiments, the scope of the present invention is not limited to these embodiments. One skilled in the art may find variations of these preferred embodiments which, nevertheless, fall within the spirit of the present invention, whose scope is defined by the claims set forth below.

### 6. EXAMPLE

### Candidate Prokaryotic Genetic Regulatory Network

The following example of a candidate genetic regulatory network is provided for purposes of illustration, and not limitation. The principles can be readily applied using materials and methods well known in the art to other biological systems, including, but not limited to, eukaryotic cell cultures, tissue samples, and organisms, including transgenic animals.

A candidate genetic regulatory network for analysis in accordance with the invention is described in Babitzke et al., 1992, Journal of Bacteriology 174: 2059-2064, which is hereby incorporated by reference in its entirety. This reference describes a set of genes in the prokaryotic organism Bacillus subtilis which are involved in aromatic amino acid biosynthesis, and are regulated by tryptophan. Thus, the physiological state of a culture of Bacillus subtilis can be analyzed by measuring the expression level of the members of this candidate genetic regulatory network under a given physiological state. For example, the culture can be grown in the absence of tryptophan. The expression levels of mRNA in the cells can be analyzed by harvesting mRNA and hybridizing the mRNA to a nucleotide array comprising appropriate nucleotide sequences as described in Section 5.2 above, using methods described in U.S. Pat. No. 5,837,832. These expression levels can then be compared to culture of Bacillus subtilis grown in the presence of abundant tryptophan. Inclusion of nucleotide sequences in the nucleotide array corresponding to the genes identified in FIG. 3 of Babitzke et al. can be used to confirm the network relationships and regulatory effects of the genes shown therein. In addition, inclusion of a vast plurality of other nucleotide sequences afforded by the use of nucleotide array chips can be used to identify other potential members of the genetic regulatory network.

Hybridization signals that specifically change intensity under one condition (e.g., +tryptophan) represent a specific change in expression as a result of the physiological perturbation of adding tryptophan. The genes whose expression level changes as a result of the tryptophan-induced perturbation are designated as damaged. The number of damaged genes is then analyzed using the numerical models for damage in a perturbed genetic regulatory network described in Section 5.5-5.7 above, to identify the green island that contains the genes whose expression is affected by tryptophan. This green island, and the expression levels of its individual member genes, provides snapshots of the physiological state of the Bacillus subtilis cells that are characteristic of either the presence or absence of tryptophan. Furthermore, individual members of this green island can then be identified by analyzing the sequences that are differentially expressed in response to the addition of tryptophan.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims. Various references including patent applications, patents, and other publications, are cited herein, the disclosures of which are incorporated by reference in their entireties.

What is claimed is:

1. A method for partitioning a plurality of genes into one or more groups comprising the steps of:
selecting a first one of said genes and a second one of said genes;
measuring a degree of correlation between said first gene and said second gene; and
assigning said first gene and said second gene into a same one of said groups if said degree of correlation exceeds a predetermined threshold.

2. The method for partitioning a plurality of genes as in claim 1, further comprising the step of repeating said selecting a first one and a second one of said genes step, said measuring a degree of correlation step and said assigning step for at least one other pair of genes selected from said plurality of genes.

3. The method for partitioning a plurality of genes of claim 1, wherein said measuring a degree of correlation step comprises the steps of:
observing a state of each of a first gene and a second gene; and
computing said degree of correlation of said state of said first gene and said state of said second gene.

4. The method for partitioning a plurality of genes as in claim 3, wherein said degree of correlation represents a mutual information MI, between said first gene and said second gene.

5. The method for partitioning a plurality of genes of claim 4, wherein said mutual information is defined as:
MI=H(A)+H(B)−H(AB)
wherein,
A represents said first gene,
B represents said second gene,
H(A) represents an entropy of said first gene,
H(B) represents an entropy of said second gene, and
H(AB) represents a joint entropy of said first gene and said second gene.

6. The method for partitioning a plurality of genes of claim 5, wherein said entropy of said gene is defined as:
Σp(i)logp(i)
wherein,
i represents said state of said gene,
p(i) represents a probability that said gene is in said state i,
log represents a logarithm operation, and
Σ represents a summation over all possible ones of said states of said gene

7. The method for partitioning a plurality of genes of claim 1, wherein a state of each of said genes is represented by a Boolean variable.

8. The method for partitioning a plurality of genes of claim 7, wherein said Boolean variable has a first value if said gene is on and a second, different value if said gene is off.

9. The method for partitioning a plurality of genes of claim 3, further comprising the preliminary step of identifying one or more of said plurality of genes that have a changing state.

10. The method for partitioning a plurality of genes of claim 9, wherein said first one and said second one of said genes are selected from said identified one or more of said plurality of genes.

11. The method for partitioning a plurality of genes of claim 1, wherein said state of each of said genes is represented by a multi-valued variable indicating an activity of said gene.

12. A system for partitioning a plurality of genes into one or more groups comprising:
a programmed computer comprising a memory having at least one region storing computer executable program code and a processor for executing the program code stored in said memory, wherein the program code includes:
code to select a first one of said genes and a second one of said genes;
code to measure a degree of correlation between said first gene and said second gene; and
code to assign said first gene and said second gene into a same one of said groups if said degree of correlation exceeds a predetermined threshold.

13. The system for partitioning a plurality of genes into one or more groups of claim 12, wherein the program code further includes:
code to define a state for each of said plurality of genes.

14. A system for partitioning a plurality of genes into one or more groups of claim 13, wherein the program code further includes:
code to receive said state of said first gene and said second gene from an RNA chip; and
code to compute said degree of correlation of said state of said first gene and said state of said second gene.

15. A method for partitioning a plurality of genes into one or more groups comprising the steps of:
defining a state for each of said genes;
selecting at least one of said genes;
initiating a perturbation on said selected gene to change said state of said selected gene; and
identifying zero or more of said genes that experience a change in said state in response to said perturbation.

16. The method for partitioning a plurality of genes of claim 15, further comprising the step of repeating said selecting at least one of said genes step, said initiating a perturbation step and said identifying zero or more of said genes that experience a change step.

17. The method for partitioning a plurality of genes of claim 15, wherein said initiating a perturbation step comprises the step of:
turning said selected gene on via an exogenous promoter.

18. The method for partitioning a plurality of genes of claim 15, wherein said initiating a perturbation step comprises the step of cloning at least one enhancer that is upstream from said selected gene.

19. A method for partitioning a plurality of genes into one or more groups comprising the steps of:
observing a state of said genes;
assigning at least one of said genes to a set; and
identifying a number of patterns of said state of said genes in said set.

20. The method for partitioning a plurality of genes of claim 19, further comprising the steps of:
assigning at least a second of said genes to said set; and
identifying a number of patterns of said state of said genes in said set.

21. The method according to claim 20, further comprising the step of assigning a multi-valued variable to represent said state of each gene.

22. The method according to claim 21, wherein said patterns represent combinations of said multi-valued variable.

23. The system for partitioning a plurality of genes into one or more groups comprising:
a programmed computer comprising a memory having at least one region storing executable program code and a processor for executing the program code stored in said memory, wherein the program code includes:
code to assign at least one of said genes to a set; and
code to identify a number of patterns of said state of said genes in said set.

24. The system for partitioning a plurality of genes into one or more groups of claim 23, wherein the program code further includes:
code to assign at least a second of said genes to said set; and
code to identify a number of patterns of said state of said genes in said set.

25. A method of determining characteristics of a plurality of genes comprising the steps of:
partitioning said plurality of genes into one or more groups;
defining a state for each of said groups; and
determining the number of steady state values of said state of said groups.

26. Computer executable software code stored on a computer readable medium, the code for partitioning a plurality of genes into one or more groups comprising:
code to select a first one of said genes and a second one of said genes;
code to determine a mutual information between said first gene and said second gene; and
code to assign said first gene and said second gene into a same one of said groups if said mutual information exceeds a predetermined threshold.
